(19) United States
(12) Patent Application Publication
(10) Pub. No.: US 2025/0026757 A1
(43) Pub. Date: Jan. 23, 2025

(54) Bicyclic Compounds for the Control of Invertebrate Pests

(71) Applicant: BASF SE, Ludwigshafen (DE)

(72) Inventors: Pulakesh Maity, Navi Mumbai (IN); Rizwan Shabbir Shaikh, Navi Mumbai (IN); Birte Schroeder, Limburgerhof (DE); Ashokkumar Adisechan, Navi Mumbai (IN)

(21) Appl. No.: 18/709,945

(22) PCT Filed: Nov. 8, 2022
(86) PCT No.: PCT/EP2022/081036

§ 371 Date: May 14, 2024

(30) Foreign Application Priority Data
Nov. 19, 2021 (EP) 21209300.9
Nov. 19, 2021 (EP) 21209301.7

(51) Int. Cl. C07D 487/04; A01N 43/90; A01P 7/04; A61K 31/519; A61K 31/53

(52) U.S. Cl. CPC C07D 487/04; A01N 43/90; A01P 7/04; A61K 31/519; A61K 31/53

(57) ABSTRACT

The invention relates to compounds of formula (I) wherein the variables have the meanings as defined in the specification, to compositions comprising them, to active compound combinations comprising them, and to their use for protecting growing plants and animals from attack or infestation by invertebrate pests, furthermore, to seed comprising such compounds.

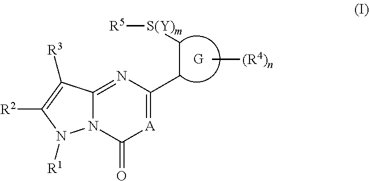

The invention relates to compounds of formula I

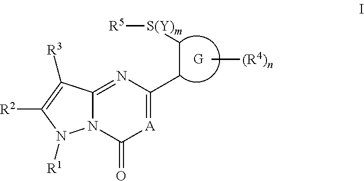

wherein
R¹ is C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkyl-C₁-C₄-alkyl, or C₃-C₆-cycloalkoxy-C₁-C₄-alkyl, which are unsubstituted or halogenated; phenyl or benzyl, wherein the rings are unsubstituted or substituted with R^{F};
   R^{F} is halogen, OH, CN, NO₂, SCN, SF₅, C₁-C₆ alkyl, C₁-C₆ alkoxy, C₂-C₆-alkenyl, C₂-C₆ alkynyl, C₁-C₆ alkoxy-C₁-C₄ alkyl, C₁-C₆ alkoxy-C₁-C₄ alkoxy, C₃-C₆ cycloalkyl, C₃-C₆-cycloalkoxy, C₃-C₆ cycloalkyl-C₁-C₄ alkyl, C₃-C₆-cycloalkoxy-C₁-C₄ alkyl, which groups are unsubstituted or substituted with halogen;
R² is H, halogen, C₁-C₆-alkyl, C₂-C₆-alkenyl, C₂-C₆-alkynyl, C₁-C₆-alkoxy, C₁-C₆-alkoxy-C₁-C₆-alkyl, C₃-C₆-cycloalkyl, C₃-C₆-cycloalkoxy, C₁-C₆-sulfenyl, C₁-C₆-sulfinyl, or C₁-C₆-sulfonyl, which groups are unsubstituted or halogenated; phenyl or benzyl, wherein the rings are unsubstituted or substituted with R^{F};
R³ is H, halogen, C₁-C₆-alkyl, C₁-C₆-alkoxy, C₃-C₆-cycloalkyl, which are unsubstituted or substituted with R^{F}; or
   phenyl or benzyl, wherein the aromatic ring of the aforementioned groups may be unsubstituted or substituted with R^{F};
G is phenyl or 6-membered hetaryl;
R⁴ is H, halogen, C(CN)R⁴¹R⁴², C(R⁴⁴)═N—OR⁴³, C(R⁴⁴)═N—N(R⁴⁵R⁴⁶), C(O)R⁴⁴, N═S(O)(R⁴¹R⁴²), N(R⁴³)C(O)R⁴⁴, N(R⁴³)C(═N—OR⁴³)R⁴⁴; OC(CN)R⁴¹R⁴², C₁-C₆-alkyl, C₁-C₆-alkoxy, C₃-C₆-cycloalkyl, C₂-C₆-alkenyl, C₂-C₆-alkynyl, phenyl, or six-membered hetaryl, which groups are unsubstituted or substituted with R^{G};
   R^{G}H, halogen, CN, NO₂, C₁-C₄-alkyl, C₁-C₄-haloalkyl, C₁-C₄-alkoxy, C₁-C₄-haloalkoxy, N(R¹²R¹³), S(O)ₘ—C₁-C₄-haloalkyl, C₁-C₄-alkylcarbonyl, or C₁-C₄-haloalkylcarbonyl; or two groups R^{G} bonded to two adjacent atoms form a 4- to 6-membered carbo- or heterocyclic ring which is unsubstituted or partially or fully substituted with R^{F};
   R⁴¹, R⁴² are independently H, CN, C₁-C₆-alkyl, C₁-C₆-haloalkyl, C₃-C₆-cycloalkyl, C₁-C₄-alkoxy, C₁-C₄-alkoxy-C₁-C₄-alkyl, S(O)ₘ—C₁-C₄-alkyl, C₁-C₄-alkyl-S(O)ₘ—C₁-C₄-alkyl, or C₁-C₄-alkoxycarbonyl;
   R⁴¹ and R⁴² may also form together with the carbon atom to which they are bound, a C₃-C₆-cycloalkyl which is unsubstituted or substituted with halogen, C₁-C₄-alkyl, C₁-C₄-haloalkyl, C₁-C₄-alkoxy, or C₁-C₄-haloalkoxy;
   R⁴³ is H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen and/or CN;
      phenyl or benzyl, which groups are unsubstituted or substituted with R^{F};
   R⁴⁴ is H, CN, OH, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen;
      phenyl or benzyl, which groups are unsubstituted or substituted with R^{F};
   R⁴⁵, R⁴⁶ are independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, C₁-C₆-alkyl-carbonyl, C₁-C₆-alkoxy-carbonyl, which groups are unsubstituted or substituted with halogen;
      phenyl or benzyl, which groups are unsubstituted or substituted with R^{F};
   NR⁴⁵R⁴⁶ may also form an N-bound, saturated 5- to 8-membered heterocycle, which additionally to the nitrogen may have 1 or 2 further heteroatoms selected from O, S(O)ₘ, and N—R′, wherein R′ is H or C₁-C₆-alkyl, and which heterocycle is unsubstituted or substituted with halogen, C₁-C₄-alkyl, C₁-C₄-haloalkyl, C₁-C₄-alkoxy, or C₁-C₄-haloalkoxy;
A is CH, CR^{A}, or N;
   R^{A} is halogen, C₁-C₄-alkyl, C₃-C₆-cycloalkyl, C₁-C₆-haloalkyl, C₁-C₆-halocycloalkyl, OR⁴³, S(O)ₘ—R⁴³; wherein rings are unsubstituted or substituted with R⁴²;
m is 0, 1, or 2
Y is O or NR^{Y};
   R^{Y} is H or C₁-C₄-alkyl;
R⁵ is C₁-C₄-alkyl, C₃-C₆-cycloalkyl, C₃-C₆-cycloalkyl-C₁-C₄-alkyl;
and the N-oxides, tautomers, stereoisomers and agriculturally or veterinarily acceptable salts thereof.

The invention also provides agricultural compositions comprising at least one compound of formula I, a stereoisomer or tautomer thereof and/or an agriculturally acceptable salt thereof and at least one liquid and/or solid carrier, especially at least one inert liquid and/or solid agriculturally acceptable carrier.

The invention also provides a veterinary composition comprising at least one compound of formula I, a stereoisomer thereof and/or a veterinarily acceptable salt thereof and at least one liquid and/or solid carrier, especially at least one inert veterinarily liquid and/or solid acceptable carrier.

The invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The invention also relates to plant propagation material, in particular seed, comprising at least one compound of formula I and/or an agriculturally acceptable salt thereof.

The invention further relates to a method for treating or protecting an animal from infestation or infection by parasites which comprises bringing the animal in contact with a parasiticidally effective amount of a compound of formula I or a veterinarily acceptable salt thereof. Bringing the animal in contact with the compound I, its salt or the veterinary composition of the invention means applying or administering it to the animal.

EP3257853 and WO2017167832 describe structurally related active compounds. These compounds are mentioned to be useful for combating invertebrate pests.

Nevertheless, there remains a need for highly effective and versatile agents for combating invertebrate pests. It is therefore an object of the invention to provide compounds having a good pesticidal activity and showing a broad activity spectrum against a large number of different invertebrate pests, especially against difficult to control pests, such as insects.

It has been found that these objects can be achieved by compounds of formula I as depicted and defined below, and by their stereoisomers, salts, tautomers and N-oxides, in particular their agriculturally acceptable salts.

### Preparation Methods

The compounds of formula (I) can be prepared by standard methods of organic chemistry. If certain derivatives cannot be prepared by the processes outlined below, they can be obtained by derivatization of other compounds of formula (I) that are accessible by these methods. The definition of variables for the following compounds and intermediates is—unless otherwise provided—as defined for formula (I). The preferred meanings of variables as defined herein is also valid for the following formulae (II) to (XII).

Compounds I can be obtained by oxidation of a compound II with a suitable oxidizing agent III. The oxidation can be effected under standard conditions known from literature.

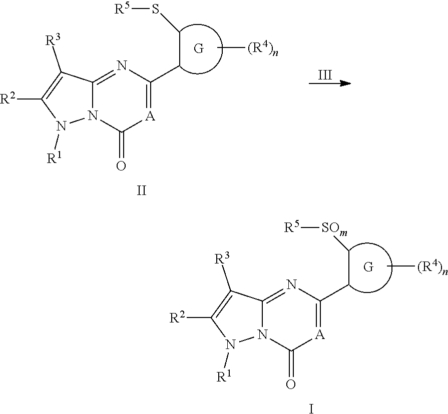

This transformation is usually carried out at temperatures of from 0° C. to 30° C., in an inert solvent, in the presence of an oxidizing reagent like mCPBA, Na₂WO₄ or H₂O₂, as described in Chemistry—A European Journal, 2017, 23(57), 14345-14357 or Bioog. Med. Chem. Lett., 2012, 22(1), 547-552 or Green Chemistry, 2009, 11(9), 1401-1405.

Suitable solvents are halogenated hydrocarbons such as methylene chloride, chloroform, and chlorobenzene, alcohols such as methanol, ethanol, n-propanol, isopropanol, n-butanol, and tert.-butanol, preferably methylene chloride, chloroform MeOH, preferably methylene chloride. It is also possible to use mixtures of the solvents mentioned. The starting materials are generally reacted with one another in equimolar amounts. In terms of yield, it may be advantageous to employ an excess of III, based on II.

Compounds II with A being CH or CR^{A} (Formula IIA) can be obtained by reaction of a compound IIIA in presence of inorganic or organic acids, such as polyphosphoric acid (PPA) or AcOH (Reagent IV). R^{AA} is a group R^{A} or H.

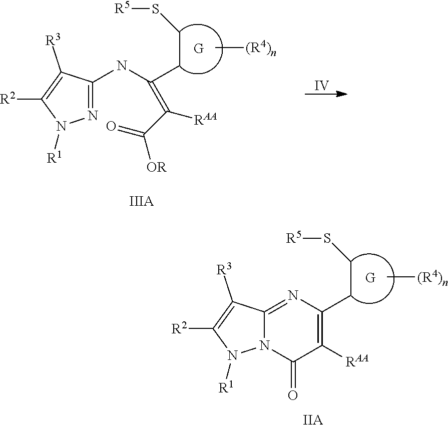

This transformation is usually carried out at temperatures of from 60° C. to 150° C., preferably from 130° C., in an inert solvent such as dioxane, in the presence of acid as described in U.S. Pat. No. 4,048,184 or Polish Journal of Chemistry, 1983, 57(10), 1219-1230.

Suitable acids and acidic catalysts are in general inorganic acids such as phosphoric acid, moreover organic acids such as formic acid, acetic acid, propionic acid, toluene sulphonic acid and trifluoro acetic acid, preferably polyphosphoric acid (PPA). The acids are generally employed in catalytic amounts; however, they can also be used in equimolar amounts, in excess or, if appropriate, as solvent. The starting materials are generally reacted with one another in equimolar amounts. In terms of yield, it may be advantageous to employ an excess of IV, based on IIIA.

Compounds IIIA can be obtained by condensation of a β-keto compound IVA with a 3-amino pyrazole V.

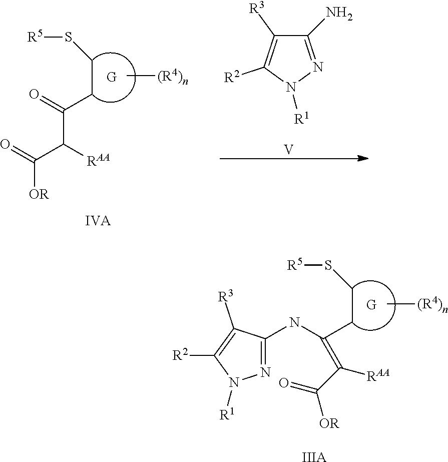

This transformation is usually carried out at temperatures of from 25° C. to 120° C., preferably at 80° C., in a protic solvent as described in Polish Journal of Chemistry, 1983, 57(10), 1219-30.

Suitable solvents are polar protic solvents like alcohols such as methanol, ethanol, n-propanol, isopropanol, n-butanol, and tert-butanol. It is also possible to use mixtures of the solvents mentioned. The starting materials are generally reacted with one another in equimolar amounts. In terms of yield, it may be advantageous to employ an excess of V, based on IVA.

Compounds V are commercially available.

Compounds IVA are obtainable from compounds VIA by alkylation with a compound VII wherein Y is a halide or another suitable nucleophilic leaving group.

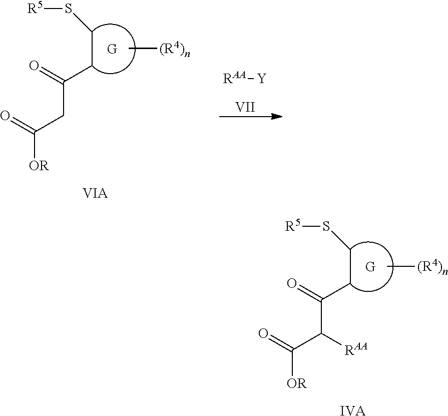

This transformation is usually carried out at temperatures of from 25° C. to 100° C., preferably at 60° C., in an inert solvent, in the presence of a base as described in Chemistry—A European Journal, 2010, 16(31), 9457-9461 or Angew. Chem. Int. Ed., 2018, 57(4), 1039-1043.

Suitable solvents are nitriles such as acetonitrile, and propionitrile. It is also possible to use mixtures of the solvents mentioned. Suitable bases are, in general, inorganic compounds, such as alkali metal and alkaline earth metal carbonates, such as Na₂CO₃, K₂CO₃, or Cs₂CO₃, preferably K₂CO₃. The bases are generally used in equimolar amounts, or even in excess. The starting materials are generally reacted with one another in equimolar amounts. In terms of yield, it may be advantageous to employ an excess of VII, based on VIA.

Compounds VII are commercially available.

Compounds VIA are obtainable from ketones VIIIA by reaction with a carbonic acid derivative, such as an ester IX.

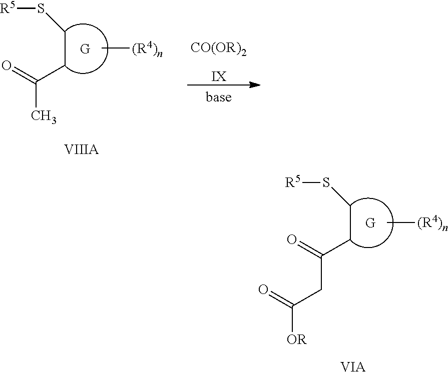

This transformation is usually carried out at temperatures of from 0° C. to 100° C., preferably from 70° C. to 100° C., in an inert solvent, in the presence of a base as described in Org. Lett., 2017, 19(23), 6344-6347 or J. Med. Chem., 1999, 42(20), 4081-4087.

Suitable solvents are aliphatic hydrocarbons such as hexane, heptane, cyclohexane, and petrol ether or ethers such as diethylether (DEE), tert-butylmethylether (TBME), dioxane, or tetrahydrofuran (THF), preferably heptane. It is also possible to use mixtures of the solvents mentioned. Suitable bases are, in general, inorganic compounds, such as alkali metal and alkaline earth metal hydrides, such as NaH, KH and CaH₂, particularly preference is given to alkali metal hydrides such as NaH. The bases are generally be used in equimolar amounts, or in excess. The starting materials are generally reacted with one another in equimolar amounts. In terms of yield, it may be advantageous to employ an excess of IX, based on VIIIA.

Ketones VIIIA are obtainable from ketones X by substitution with an alkylthiolate XI, as known from literature (cf. WO2015091945). In formula X “Hal” denotes a halogen atom, preferably Cl or F, in formula XI M denotes a cation, such as an alkali metal.

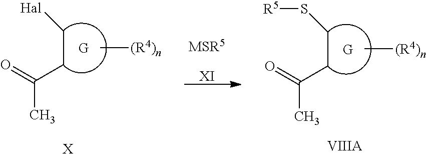

This transformation is usually carried out at temperatures of from 25° C. to 60° C., preferably at 20-25° C., in an inert solvent, in the presence of a base, following the conditions as described in WO2015091945 or WO2018095795.

Suitable solvents are ethers such as DEE, TBME, dioxane, and THF, moreover dimethyl sulphoxide (DMSO), dimethyl formamide (DMF), or dimethylacetamide (DMA), preferably DMF, or ethers such as THF. It is also possible to use mixtures of the solvents mentioned.

Suitable bases are, in general, inorganic compounds, such as alkali metal hydroxides, such as NaOH, KOH, or alkali metal hydrides, such as NaH, KH, or alkali metal and alkaline earth metal carbonates, such as Na₂CO₃, K₂CO₃, or Cs₂CO₃, or Na or K tert-butoxide. The bases are generally in equimolar amounts or even in excess.

The starting materials are generally reacted with one another in equimolar amounts. In terms of yield, it may be advantageous to employ an excess of XI, based on X.

Compounds II with A being N (Formula IIB) can be obtained by reaction of a compound IIIB with XII (ethyl (pivaloyloxy) carbamate) as the carbamylation reagent wherein OR is a leaving group such as alkoxy.

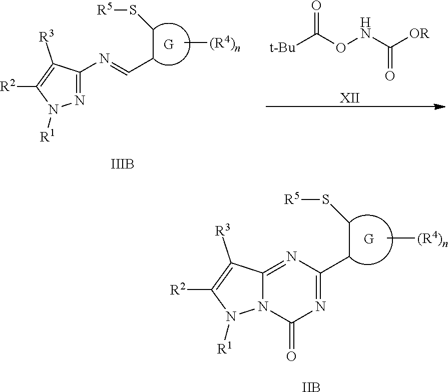

This transformation is usually carried out at temperatures of from 25° C. to 100° C., preferably at 60° C., in an inert solvent, in the presence of a catalyst in a mild acidic condition, as described in Org. Lett., 2020, 22 (22), 8993-8997.

Suitable solvents are halogenated hydrocarbons such as methylene chloride, dichloroethane, chloroform, and chlorobenzene, preferably dichloroethane. It is also possible to use mixtures of the solvents mentioned. Suitable acids are in general organic acids such as acetic acid, propionic acid, toluene sulphonic acid and trifluoro acetic acid, preferably pivalic acid. The acids are generally employed in catalytic amounts; however, they can also be used in equimolar amounts, even in excess. The starting materials are generally reacted with one another in equimolar amounts. In terms of yield, it may be advantageous to employ an excess of XII, based on IIIB.

Compounds XII are commercially available or known from literature cited.

Compounds IIIB can be obtained by condensation of an aldehyde IVB with a 3-amino pyrazole V.

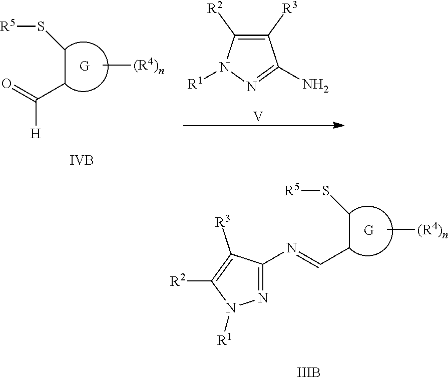

This transformation is usually carried out at temperatures of from 25° C. to 70° C., preferably at 70° C., in a protic solvent as described in WO2020075706.

Suitable solvents are in general alcohols such as methanol, ethanol, n-propanol, isopropanol, n-butanol, and tert-butanol, preferably ethanol. It is also possible to use mixtures of the solvents mentioned. The starting materials are generally reacted with one another in equimolar amounts. In terms of yield, it may be advantageous to employ an excess of V, based on IVB.

Compounds V are commercially available.

Aldehydes IVB are obtainable from aldehydes XII by substitution with an alkylthiolate XI, as outlined above (cf. WO2000034258). In formula XII “Hal” denotes a halogen atom, preferably Cl or F.

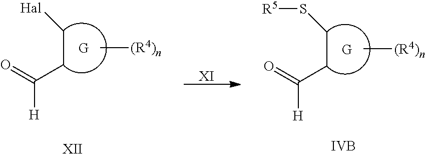

The starting materials are generally reacted with one another in equimolar amounts. In terms of yield, it may be advantageous to employ an excess of XI, based on XII.

Aldehydes XII are commercially available or known from literature cited.

Compounds I with Y being NR¹²¹ are obtainable from the analogous thioether compounds of formula II by with a suitable oxidizing agent XIII.

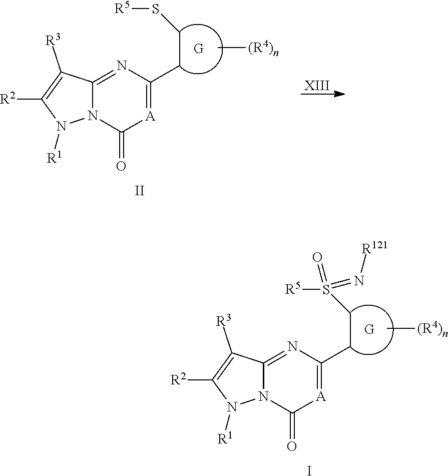

This transformation is usually carried out at temperatures of from 0° C. to 25° C., preferably at 20-25° C., in a protic solvent, in the presence of an amine source and an oxidant like PhI(OAc)₂, following the analogy to method as described in Org. Lett., 2020, 22(19), 7470-7474 or Chemistry Select, 2017, 2(4), 1620-1624

Suitable solvents are polar protic solvents like alcohols such as methanol, ethanol, n-propanol, isopropanol, n-butanol, and tert-butanol, preferably methanol. It is also possible to use mixtures of the solvents mentioned. Suitable amine sources are like aqueous ammonia, ammonium formate, (NH₄)₂CO₃, NH₄F, ammonium oxalate, preferably (NH₄)₂CO₃. Suitable oxidants are in general hypervalent iodine reagent such as PhI(OAc)₂ or PhI(TFA)₂. The oxidants are generally used in in equimolar amounts, or in excess.

The reaction mixtures are worked up in a customary manner, for example by mixing with water, extracting with an appropriate organic solvent, separating the phases and, if appropriate, chromatographic purification of the crude products. Some of the intermediates and end products are obtained in the form of colourless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

However, if the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the pest to be controlled.

The organic moieties groups mentioned in the above definitions of the variables are—like the term halogen—collective terms for individual listings of the individual group members. The prefix COₙ—Cₘ indicates in each case the possible number of carbon atoms in the group.

The term “substituted with”, e.g. as used in “partially, or fully substituted with” means that one or more, e.g. 1, 2, 3, 4 or 5 or all of the hydrogen atoms of a given radical have been replaced by one or more, same or different substituents, such as a halogen. Accordingly, for substituted cyclic moieties, e.g. 1-cyanocyclopropyl, one or more of the hydrogen atoms of the cyclic moiety may be replaced by one or more, same or different substituents.

If it is indicated that a variable that may occur more than once In formula I may be selected from several alternatives, it means that each may be independently selected from said alternatives. Accordingly, the phrase “R⁴ is H, halogen, C(CN)R⁴¹R⁴², C(R⁴⁴)═N—OR⁴³” etc means that each R″ is independently selected from H, halogen C(CN)R⁴¹R⁴², C(R⁴⁴)═N—OR⁴³ etc.

The term “halogen” denotes in each case fluorine, bromine, chlorine, or iodine, in particular fluorine, chlorine, or bromine.

The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl (Me), ethyl (Et), n-propyl (n-Pr), iso-propyl (iPr), n-butyl, 2-butyl, iso-butyl, tert-butyl (tBu), n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂—OC₂H₅, 2-(methoxy)ethyl, and 2-(ethoxy)ethyl.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms. Preferred haloalkoxy moieties include C₁-C₄-haloalkoxy, in particular C₁-C₂-fluoroalkoxy, such as fluoromethoxy, difluoromethoxy, trifluoro-methoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2dichloro-2-fluoroethoxy, 2,2,2-trichloroethoxy, penta-fluoroethoxy and the like.

The term “alkylthio “(alkylsulfanyl: S-alkyl)” as used herein refers to a straight-chain or branched saturated alkyl group having 1 to 10 carbon atoms, preferably 1 to 4 carbon atoms (═C₁-C₄-alkylthio), more preferably 1 to 3 carbon atoms, which is attached via a sulfur atom.

The term “haloalkylthio” as used herein refers to an alkylthio group as mentioned above wherein the hydrogen atoms are partially or fully substituted by fluorine, chlorine, bromine and/or iodine.

The term “alkylsulfinyl” (alkylsulfoxyl: S(═O)-alkyl), as used herein refers to a straight-chain or branched saturated alkyl group (as mentioned above) having 1 to 10 carbon atoms, preferably 1 to 4 carbon atoms (═C₁-C₄-alkylsulfinyl), more preferably 1 to 3 carbon atoms bonded through the sulfur atom of the sulfinyl group at any position in the alkyl group.

The term “haloalkylsulfinyl” as used herein refers to an alkylsulfinyl group as mentioned above wherein the hydrogen atoms are partially or fully substituted by fluorine, chlorine, bromine and/or iodine.

The term “alkylsulfonyl” (S(═O)₂-alkyl) as used herein refers to a straight-chain or branched saturated alkyl group having 1 to 10 carbon atoms, preferably 1 to 4 carbon atoms (═C₁-C₄-alkylsulfonyl), preferably 1 to 3 carbon atoms, which is bonded via the sulfur atom of the sulfonyl group at any position in the alkyl group.

The term “haloalkylsulfonyl” as used herein refers to an alkylsulfonyl group as mentioned above wherein the hydrogen atoms are partially or fully substituted by fluorine, chlorine, bromine and/or iodine.

The term “alkylcarbonyl” refers to an alkyl group as defined above, which is bonded via the carbon atom of a carbonyl group (C═O) to the remainder of the molecule.

The term “haloalkylcarbonyl” refers to an alkylcarbonyl group as mentioned above, wherein the hydrogen atoms are partially or fully substituted by fluorine, chlorine, bromine and/or iodine.

The term “alkoxycarbonyl” refers to an alkylcarbonyl group as defined above, which is bonded via an oxygen atom to the remainder of the molecule.

The term “haloalkoxycarbonyl” refers to an alkoxycarbonyl group as mentioned above, wherein the hydrogen atoms are partially or fully substituted by fluorine, chlorine, bromine and/or iodine.

The term “alkenyl” as used herein denotes in each case a singly unsaturated hydrocarbon radical having usually 2 to 10, frequently 2 to 6, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “haloalkenyl” as used herein refers to an alkenyl group as defined above, wherein the hydrogen atoms are partially or totally replaced with halogen atoms.

The term “alkynyl” as used herein denotes in each case a singly unsaturated hydrocarbon radical having usually 2 to 10, frequently 2 to 6, preferably 2 to 4 carbon atoms, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “haloalkynyl” as used herein refers to an alkynyl group as defined above, wherein the hydrogen atoms are partially or totally replaced with halogen atoms.

The term “cycloalkyl” as used herein and in the cycloalkyl moieties of cycloalkoxy and cycloalkylthio denotes in each case a monocyclic cycloaliphatic radical having usually from 3 to 10 or from 3 to 6 carbon atoms, such as cyclopropyl (cC₃H₅), cyclobutyl (cC₄H₇), cyclopentyl (cC₅H₉), cyclohexyl (cC₆H₁₁), cycloheptyl, cyclooctyl, cyclononyl and cyclodecyl or cyclopropyl, cyclobutyl, cyclopentyl and cyclohexyl.

The term “halocycloalkyl” as used herein and in the halocycloalkyl moieties of halocycloalkoxy and halocycloalkylthio denotes in each case a monocyclic cycloaliphatic radical having usually from 3 to 10 C atoms or 3 to 6 C atoms, wherein at least one, e.g. 1, 2, 3, 4 or 5 of the hydrogen atoms, are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclopropyl, 1- and 2-chlorocyclopropyl, 1,2-, 2,2- and 2,3-dichlorocyclopropyl, 1,2,2-trichloro-cyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “halocycloalkenyl” as used herein and in the halocycloalkenyl moieties of halocycloalkenyloxy and halocycloalkenylthio denotes in each case a monocyclic singly unsaturated non-aromatic radical having usually from 3 to 10, e.g. 3 or 4 or from 5 to 10 carbon atoms, preferably from 3- to 8 carbon atoms, wherein at least one, e.g. 1, 2, 3, 4 or 5 of the hydrogen atoms, are replaced by halogen, in particular by fluorine or chlorine. Examples are 3,3-difluorocyclopropen-1-yl and 3,3-dichlorocyclopropen-1-yl.

The term “cycloalkenylalkyl” refers to a cycloalkenyl group as defined above which is bonded via an alkyl group, such as a C₁-C₅-alkyl group or a C₁-C₄-alkyl group, in particular a methyl group (=cycloalkenylmethyl), to the remainder of the molecule.

The term “carbocycle” or “carbocyclyl” includes in general a 3- to 12-membered, preferably a 3- to 8-membered or a 5- to 8-membered, more preferably a 5- or 6-membered mono-cyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above.

The term “heterocycle” or “heterocyclyl” includes in general 3- to 12-membered, preferably 3- to 6-membered, in particular 6-membered monocyclic heterocyclic non-aromatic radicals. The heterocyclic non-aromatic radicals usually comprise 1, 2, 3, 4 or 5, preferably 1, 2 or 3 heteroatoms selected from N, O, and S as ring members, wherein S-atoms as ring members may be present as S, SO, or SO₂. Examples of 5- or 6-membered heterocyclic radicals comprise saturated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxo-thiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S. oxothiopyranyl, S-dioxothiopyranyl, dihydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-only, and the like.

The term “hetaryl” includes monocyclic 5- or 6-membered heteroaromatic radicals comprising as ring members 1, 2, 3 or 4 heteroatoms selected from N, O, and S. Examples of 5- or 6-membered heteroaromatic radicals include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl. The term “hetaryl” also includes bicyclic 8 to 10-membered heteroaromatic radicals comprising as ring members 1, 2 or 3 heteroatoms selected from N, O, and S, wherein a 5- or 6-membered heteroaromatic ring is fused to a phenyl ring or to a 5- or 6-membered heteroaromatic radical. Examples of a 5- or 6-membered heteroaromatic ring fused to a phenyl ring or to a 5- or 6-membered heteroaromatic radical include benzofuranyl, benzo-thienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like. These fused hetaryl radicals may be bonded to the remainder of the molecule via any ring atom of 5- or 6-membered heteroaromatic ring or via a carbon atom of the fused phenyl moiety.

The terms “heterocyclylalkyl” and “hetarylalkyl” refer to heterocyclyl or hetaryl, respectively, as defined above which are bonded via a C₁-C₅-alkyl group or a C₁-C₄-alkyl group, in particular a methyl group (=heterocyclylmethyl or hetarylmethyl, respectively), to the remainder of the molecule.

The term “arylalkyl” and “phenylalkyl” refer to aryl as defined above and phenyl, respectively, which are bonded via C₁-C₅-alkyl group or a C₁-C₄-alkyl group, in particular a methyl group (=arylmethyl or phenylmethyl), to the remainder of the molecule, examples including benzyl, 1-phenylethyl, 2-phenylethyl, 2-phenoxyethyl etc.

The terms “alkylene”, “cycloalkylene”, “heterocycloalkylene”, “alkenylene”, “cycloalkenylene”, “heterocycloalkenylene” and “alkynylene” refer to alkyl, cycloalkyl, heterocycloalkyl, alkenyl, cycloalkenyl, heterocycloalkenyl and alkynyl as defined above, respectively, which are bonded to the remainder of the molecule, via two atoms, preferably via two carbon atoms, of the respective group, so that they represent a linker between two moieties of the molecule.

In a particular embodiment, the variables of the compounds of the formula I have the following meanings, these meanings, both on their own and in combination with one another, being particular embodiments of the compounds of the formula I.

Embodiments and preferred compounds of the invention for use in pesticidal methods and for insecticidal application purposes are outlined in the following paragraphs.

With respect to the variables, the particularly preferred embodiments of the intermediates correspond to those of the compounds of the formula I.

Preferably R¹ is C₁-C₆-alkyl, or C₃-C₆-cycloalkyl, more preferably C₁-C₃-alkyl or cyclopropyl, such as CH₃ or cyclopropyl, especially CH₃.

Preferably R² is C₁-C₄-haloalkyl, or C₃-C₆-cycloalkyl, more preferably C₁-C₃-haloalkyl or cyclopropyl, such as CHF₂, CF₃, CF₂CH₃, or cyclopropyl, in particular CF₃ or cyclopropyl. In another embodiment, R² is H, C₁-C₄-haloalkyl, or C₃-C₆-cycloalkyl, more preferably H, C₁-C₃-haloalkyl or cyclopropyl.

Preferably R³ is H. In another embodiment, R³ is H or phenyl, wherein the phenyl is unsubstituted or substituted with halogen, preferably H or phenyl substituted with halogen.

R⁵ is preferably C₁-C₄-alkyl, more preferably C₁-C₃-alkyl, in particular ethyl.

A is preferably CH or CR^{A}, preferably wherein R^{A} is C₁-C₃-alkyl or halogen, in particularly CH or C—CH₃. Such compounds correspond to Formula IA

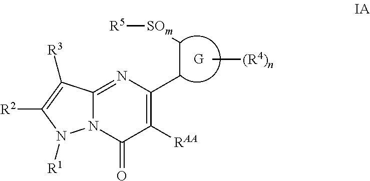

In another embodiment A is N. Such compounds correspond to formula IB.

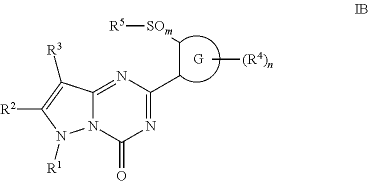

G is preferably a six-membered hetaryl, such as pyridyl, preferably 2-pyridyl.

In another embodiment G is phenyl.

Such compounds correspond to formula IG, wherein Q is CH or N:

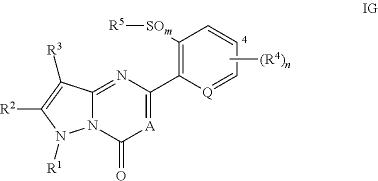

R⁴ is preferably in para-position (relative to the bicyclic attachment of the 6-membered ring), and is preferably selected from halogen, such as F, Cl, Br; CN; N(R⁴³)C(O)R⁴⁴, N═S(O)(CH₃)₂; C₁-C₄-alkyl, C₃-C₆-cycloalkyl, or C₁-C₄-alkoxy, both unsubstituted or substituted with CN or halogen; C₂-C₄-alkenyl or C₂-C₄-alkynyl both unsubstituted or substituted with C₃-C₆-cycloalkyl;
phenyl or pyridyl which aromatics are unsubstituted or substituted with R^{G}, which are preferably selected from halogen, CN, C₁-C₄-alkyl, C₁-C₄-alkoxy, C₁-C₄-haloalkyl, C₁-C₄-haloalkoxy; or two groups R^{G} bonded to two adjacent ring atoms form a 4- to 6-membered carbo- or heterocyclic ring which is unsubstituted or partially or fully substituted with R^{F}, which is preferably halogen.

In another embodiment, R⁴ is selected from H, halogen, such as F, Cl, Br; CN; N═S(O)(CH₃)₂;
phenyl or pyridyl which are unsubstituted or substituted with R^{G}, which is preferably selected from halogen, CN, C₁-C₄-alkoxy, C₁-C₄-haloalkoxy;
or two groups R^{G} bonded to two adjacent ring atoms form a 4- to 6-membered carbo- or heterocyclic ring which is unsubstituted or partially or fully substituted with R^{F}, which is preferably halogen.

In another embodiment, R⁴ is selected from H, halogen, such as F, Cl, Br; CN; N═S(O)(CH₃)₂;
phenyl or pyridyl which are substituted with R^{G} selected from halogen, CN, or C₁-C₃-haloalkoxy;
or two groups R^{G} bonded to two adjacent ring atoms form a 5-membered carbo- or heterocyclic ring which is unsubstituted or partially or fully substituted with R^{F}, which is preferably halogen.

In another embodiment, R⁴ is selected from H, halogen, such as F, Cl, Br; CN; N═S(O)(CH₃)₂;
phenyl or pyridyl which are substituted with R^{G} selected from halogen, CN, or C₁-C₃-haloalkoxy;
or two groups R^{G} bonded to two adjacent ring atoms form a 5-membered carbo- or heterocyclic ring containing two O-atoms as ring members, which carbo- or heterocyclic ring is substituted with R^{F}, which is preferably halogen.

The index n is preferably 1 or 2, particularly 1. Y is preferably O. Index m is preferably 2.

In particular with a view to their use, preference is given to the compounds of formula I com-piled in the tables below, which compounds correspond to formula I.G*. Each of the groups mentioned for a substituent in the tables is furthermore per se, independently of the combination in which it is mentioned, a particularly preferred aspect of the substituent in question.

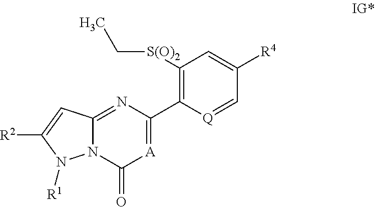

### Table 1

Compounds of formula IG* in which A is CH, R¹ is CH₃, R² is CF₃ and the combination of Q and R⁴ for a compound corresponds in each case to one row of Table A

### Table 2

Compounds of formula IG* in which A is C—CH₃, R¹ is CH₃, R² is CF₃ and the combination of Q and R⁴ for a compound corresponds in each case to one row of Table A

### Table 3

Compounds of formula IG* in which A is N, R¹ is CH₃, R² is CF₃ and the combination of Q and R⁴ for a compound corresponds in each case to one row of Table A

### Table 4

Compounds of formula IG* in which A is C—Br, R¹ is CH₃, R² is CF₃ and the combination of Q and R⁴ for a compound corresponds in each case to one row of Table A

### Table 5

Compounds of formula G* in which A is CH, R¹ is c-C₃H₅, R² is CF₃ and the combination of Q and R⁴ for a compound corresponds in each case to one row of Table A

### Table 6

Compounds of formula IG* in which A is C—CH₃, R¹ is c-C₃H₅, R² is CF₃ and the combination of Q and R⁴ for a compound corresponds in each case to one row of Table A

### Table 7

Compounds of formula IG* in which A is N, R¹ is c-C₃H₅, R² is CF₃ and the combination of Q and R⁴ for a compound corresponds in each case to one row of Table A

### Table 8

Compounds of formula IG* in which A is CH, R¹ is CH₃, R² is CHF₂ and the combination of Q and R⁴ for a compound corresponds in each case to one row of Table A

### Table 9

Compounds of formula IG* in which A is C—CH₃, R¹ is CH₃, R² is CHF₂ and the combination of Q and R⁴ for a compound corresponds in each case to one row of Table A

### Table 10

Compounds of formula IG* in which A is N, R¹ is CH₃, R² is CHF₂ and the combination of Q and R⁴ for a compound corresponds in each case to one row of Table A

### Table 11

Compounds of formula G* in which A is CH, R¹ is c-C₃H₅, R² is CHF₂ and the combination of Q and R⁴ for a compound corresponds in each case to one row of Table A

### Table 12

Compounds of formula IG* in which A is C—CH₃, R¹ is c-C₃H₅, R² is CHF₂ and the combination of Q and R⁴ for a compound corresponds in each case to one row of Table A

### Table 13

Compounds of formula IG* in which A is N, R¹ is c-C₃H₅, R² is CHF₂ and the combination of Q and R⁴ for a compound corresponds in each case to one row of Table A

### Table 14

Compounds of formula IG* in which A is CH, R¹ is CH₃, R² is c-C₃H₅ and the combination of Q and R⁴ for a compound corresponds in each case to one row of Table A

### Table 15

Compounds of formula IG* in which A is C—CH₃, R¹ is CH₃, R² is c-C₃H₅ and the combination of Q and R⁴ for a compound corresponds in each case to one row of Table A

### Table 16

Compounds of formula IG* in which A is N, R¹ is CH₃, R² is c-C₃H₅ and the combination of Q and R⁴ for a compound corresponds in each case to one row of Table A

### Table 17

Compounds of formula IG* in which A is C—Br, R¹ is CH₃, R² is c-C₃H₅ and the combination of 0 and R⁴ for a compound corresponds in each case to one row of Table A

### Table 18

Compounds of formula G* in which A is CH, R¹ is c-C₃H₅, R² is c-C₃H₅ and the combination of 0 and R⁴ for a compound corresponds in each case to one row of Table A

### Table 19

Compounds of formula IG* in which A is C—CH₃, R¹ is c-C₃H₅, R² is C c-C₃H₅F₃ and the combination of Q and R⁴ for a compound corresponds in each case to one row of Table A

### Table 20

Compounds of formula IG* in which A is N, R¹ is c-C₃H₅, R² is c-C₃H₅ and the combination of Q and R⁴ for a compound corresponds in each case to one row of Table A

TABLE A

| No. | Q | R⁴ |
|---|---|---|
| A-1 | CH | H |
| A-2 | CH | F |
| A-3 | CH | Cl |
| A-4 | CH | Br |
| A-5 | CH | I |
| A-6 | CH | CN |
| A-7 | CH | CH₃ |
| A-8 | CH | C₂H₅ |
| A-9 | CH | CH₂CH₂CH₃ |
| A-10 | CH | CH(CH₃)₂ |
| A-11 | CH | OCH₃ |
| A-12 | CH | OC₂H₅ |
| A-13 | CH | OCH₂CH₂CH₃ |
| A-14 | CH | OCH(CH₃)₂ |
| A-15 | CH | CH₂F |
| A-16 | CH | CHF₂ |
| A-17 | CH | CF₃ |
| A-18 | CH | OCH₂F |
| A-19 | CH | OCHF₂ |
| A-20 | CH | OCF₃ |
| A-21 | CH | C═CHCH₃ |
| A-22 | CH | C═CH—c-C₃H₅ |
| A-23 | CH | C≡CCH₃ |
| A-24 | CH | C≡C—c-C₃H₅ |
| A-25 | CH | N═S(O)(CH₃)₂ |
| A-26 | CH | C(CN)(CH₃)₂ |
| A-27 | CH | OC(CN)(CH₃)₂ |
| A-28 | CH | N(CH₃)C(O)CH₃ |
| A-29 | CH | 4-F—C₆H₄ |
| A-30 | CH | 4-Cl—C₆H₄ |
| A-31 | CH | 4-Br—C₆H₄ |
| A-32 | CH | 4-I—C₆H₄ |
| A-33 | CH | 4-CN—C₆H₄ |
| A-34 | CH | 4-OCHF₂—C₆H₄ |
| A-35 | CH | 4-F-pyrid-3-yl |
| A-36 | CH | 4-Cl-pyrid-3-yl |
| A-37 | CH | 4-Br-pyrid-3-yl |
| A-38 | CH | 4-I-pyrid-3-yl |
| A-39 | CH | 4-CN-pyrid-3-yl |
| A-40 | CH | 2-F-4-CF₃O—C₆H₄ |
| A-41 | CH | 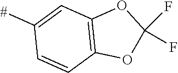 |
| A-42 | CH | 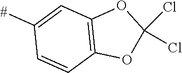 |
| A-43 | CH | 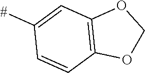 |
| A-44 | N | H |
| A-45 | N | F |
| A-46 | N | Cl |
| A-47 | N | Br |
| A-48 | N | I |
| A-49 | N | CN |
| A-50 | N | CH₃ |
| A-51 | N | C₂H₅ |
| A-52 | N | CH₂CH₂CH₃ |
| A-53 | N | CH(CH₃)₂ |
| A-54 | N | OCH₃ |
| A-55 | N | OC₂H₅ |
| A-56 | N | OCH₂CH₂CH₃ |
| A-57 | N | OCH(CH₃)₂ |
| A-58 | N | CH₂F |
| A-59 | N | CHF₂ |
| A-60 | N | CF₃ |
| A-61 | N | OCH₂F |
| A-62 | N | OCHF₂ |
| A-63 | N | OCF₃ |
| A-64 | N | C═CHCH₃ |
| A-65 | N | C═CH—c-C₃H₅ |
| A-66 | N | C≡CCH₃ |
| A-67 | N | C≡C—c-C₃H₅ |
| A-68 | N | N═S(O)(CH₃)₂ |
| A-69 | N | C(CN)(CH₃)₂ |
| A-70 | N | OC(CN)(CH₃)₂ |
| A-71 | N | N(CH₃)C(O)CH₃ |
| A-72 | N | 4-F—C₆H₄ |
| A-73 | N | 4-Cl—C₆H₄ |
| A-74 | N | 4-Br—C₆H₄ |
| A-75 | N | 4-I—C₆H₄ |
| A-76 | N | 4-CN—C₆H₄ |
| A-77 | N | 4-OCHF₂—C₆H₄ |
| A-78 | N | 4-F-pyrid-3-yl |
| A-79 | N | 4-Cl-pyrid-3-yl |
| A-80 | N | 4-Br-pyrid-3-yl |
| A-81 | N | 4-I-pyrid-3-yl |
| A-82 | N | 4-CN-pyrid-3-yl |
| A-83 | N | 2-F-4-CF₃O—C₆H₄ |
| A-84 | N | 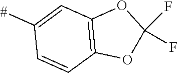 |
| A-85 | N | 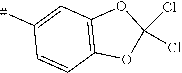 |
| A-86 | N | 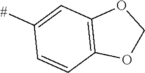 |

The “#” signifies the connection to the remaining part of formula (I).

The term “compound(s) of the invention” refers to compound(s) of formula I, or “coin-pound(s) I”, and includes their salts, tautomers, stereoisomers, and N-oxides.

The invention also relates to agrochemical compositions comprising an auxiliary and at least one compound I.

An agrochemical composition comprises a pesticidally effective amount of a compound I.

The compounds I can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International. The compositions are prepared in a known manner, e.g. described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emulsifier, dispersant, solubilizer, wetter, penetration enhancer, protective colloid, or adjuvant. Surfactants are listed in McCutcheon's, Vol. 1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-substituted fatty acid amides, amine oxides, esters, sugar-based surfactants, polymeric surfactants. Suitable cationic surfactants are qua-ternary surfactants.

The agrochemical compositions generally comprise between 0.01 and 95%, preferably between 0.1 and 90%, and most preferably between 0.5 and 75%, by weight of active substance.

The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agro-chemical composition is made up with water, buffer, and/or further auxiliaries to the desired application concentration and the ready-to-use spray liquor or the agrochemical composition according to the invention is thus obtained. Usually, 20 to 2000 liters, of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound I.

The compounds I are also suitable for use in combating or controlling animal pests. There-fore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound I.

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grape-fruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds I are also suitable for use against non-crop insect pests. For use against said non-crop pests, compounds I can be used as bait composition, gel, general insect spray, aerosol, as ultra-low volume application and bed net (impregnated or surface applied).

The term “non-crop insect pest” refers to pests, which are particularly relevant for non-crop targets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta Americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds I and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 wt %, preferably from 0.1 to 45 wt %, and more preferably from 1 to 25 wt % of at least one repellent and/or insecticide.

### Pests

The compounds of the invention are especially suitable for efficiently combating animal pests e.g. arthropods, and nematodes including:
insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;
Lepidoptera, e.g. Helicoverpa spp., Heliothis virescens, Lobesia botrana, Ostrinia nubilalis, Plutella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;
True bugs, e.g. Lygus spp., Stink bugs such as Euschistus spp., Halyomorpha halys, Nezara viridula, Piezodorus guildinii, Dichelops furcatus;
Thrips, e.g. Frankliniella spp., Thrips spp., Dichromothrips corbettii;
Aphids, e.g. Acyrthosiphon pisum, Aphis spp., Myzus persicae, Rhopalosiphum spp., Schizaphis graminum, Megoura viciae;
Whiteflies, e.g. Trialeurodes vaporariorum, Bemisia spp.;
Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp.;
Flies, e.g. Delia spp., Ceratitis capitate, Bactrocera spp., Liriomyza spp.;
Coccoidea, e.g. Aonidiella aurantia, Ferrisia virgate;
Anthropods of class Arachnida (Mites), e.g. Penthaleus major, Tetranychus spp.;
Nematodes, e.g. Heterodera glycines, Meloidogyne spp., Pratylenchus spp., Caenorhabditis elegans.

### Animal Health

The compounds I are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound I.

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound I.

The compounds of the invention are further suitable for use in combating or controlling parasites in and on animals. Furthermore, the invention relates to a method of combating or controlling parasites in and on animals, which comprises contacting the parasites with a parasitically effective amount of a compound I.

The invention also relates to the non-therapeutic use of compounds I for controlling or combating parasites. Moreover, the invention relates to a non-therapeutic method of combating or controlling parasites, which comprises applying to a locus a parasiticidally effective amount of a compound I.

The compounds I can be effective through both contact (via soil, glass, wall, bed net, carpet, blankets or animal parts) and ingestion (e.g. baits). Furthermore, the compounds I can be applied to any and all developmental stages.

The compounds I can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides felis.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds I may be applied in total amounts of 0.5 mg/kg to 100 mg/kg per day, preferably 1 mg/kg to 50 mg/kg per day.

For oral administration to warm-blooded animals, the compounds I may be formulated as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds I, preferably with 0.5 mg/kg to 100 mg/kg of animal body weight per day.

Alternatively, the compounds I may be administered to animals parenterally, e.g., by intraruminal, intramuscular, intravenous or subcutaneous injection. The compounds I may be dispersed or dissolved in a physiologically acceptable carrier for subcutaneous injection. Alternatively, the compounds I may be formulated into an implant for subcutaneous administration. In addition the compounds I may be transdermally administered to animals. For parenteral administration, the dosage form chosen should provide the animal with 0.01 mg/kg to 100 mg/kg of animal body weight per day of the compounds I.

The compounds I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds I. In addition, the compounds I may be formulated as ear tags for animals, particularly quadrupeds e.g. cattle and sheep.

Oral solutions are administered directly.

Solutions for use on the skin are trickled on, spread on, rubbed in, sprinkled on or sprayed on.

Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound I.

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight.

Preparations which are diluted before use contain the compounds acting against ectoparasites in concentrations of 0.5 to 90% by weight, preferably of 1 to 50% by weight.

Furthermore, the preparations comprise the compounds of formula I against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

### A. PREPARATION EXAMPLES

Materials: Unless otherwise noted, reagents and solvents were purchased at highest commercial quality and used without further purification. Acetonitrile (MeCN); Dry tetrahydrofuran (THF), ethylacetate (EtOAc), diethylethyer (DEE), dimethylsulfoxide (DMSO), acetone, ethanol (EtOH), benzene, dimethylformamide (DMF), diisopropylethylamine (DIPEA), hexafluorophosphate azabenzotriazole tetramethyl uronium (HATU), pyridine, and CH₂Cl₂ (DCM) were purchased from commercial providers.

The compounds were characterized by melting point determination, by NMR spectroscopy or by the mass-to-charge ratio ([m/z]) and retention time (Rₜ; [min]), as determined by mass spectrometry (MS) coupled with HPLC analysis (HPLC-MS=high performance liquid chromatography-coupled mass spectrometry) or LC analysis (LC-MS=liquid chromatography-coupled mass spectrometry).

Characterization: The compounds were characterized by coupled High Performance Liquid Chromatography with mass spectrometry (HPLC/MS).

All reactions were monitored by thin-layer chromatography (TLC) using Merck silica gel 60 F₂54 pre-coated plates (0.25 mm). Flash chromatography was carried out with Kanto Chemical silica gel (Kanto Chemical, silica gel 60N, spherical neutral, 0.040-0.050 mm, Cat.-No. 37563-84). If not otherwise indicated, ¹H NMR spectra were recorded on JEOL JNM-ECA-500 (500 MHz). Chemical shifts are expressed in ppm downfield from the internal solvent peaks for acetone-d₆ (¹H; δ=2.05 ppm) and CD₃OD (¹H; δ=3.30 ppm), and J values are given in Hertz. The following abbreviations were used to explain the multiplicities: s=singlet, d=doublet, t=triplet, q=quartet, dd=double doublet, dt=double triplet, m=multiplet, br=broad. High-resolution mass spectra were measured on a JEOL JMS-T100LP.

Method A: Agilent Eclipse Plus C18, 50×4.6 mm, ID 5 μm; Elution: A=10 mM Amm. Formate (0.1% Formic Acid), B=Acetonitrile (0.1% Formic Acid), Flow=1.2 ml/min at 30° C.; Gradient: 10% B to 100% B—3 min, hold for 1 min, 1 min—10% B. Run Time=5.01 min; MS: ESI positive; Mass range (m/z): 100-700.

Method B: LC: Shimadzu LC-30AD, ESI; Column: Kinetex EVO C18.5 μm 2.1×30 mm; Mobile phase: A: water+0.04% TFA; B: ACN+0.02% TFA; Temperature: 40° C.; Gradient: 5% B to 100% B in 2.5 min; 100% B to 5% B in 0.02 min; 5% B for 0.5 min; Flow: 0.8 mL/min; MS: ESI positive; Mass range: 100-2000.

Abbreviations: mL (milliliters); g (grams); h (hour(s)); min (minutes).

### Example 1: Synthesis of 5-(5-bromo-3-ethylsulfonyl-2-pyridyl)-1-methyl-2-(trifluoromethyl)pyrazolo[1,5-a]pyrimidin-7-one (I-1)

### Step 1: Synthesis of 5-bromo-3-ethylsulfanyl-pyridine-2-carbonitrile

To a stirred solution of 5-bromo-3-nitro-pyridine-2-carbonitrile (5.0 g) in dry THF (45 mL) and water (5 mL) mixture was added sodium ethane thiolate (2.17 g) at −10° C. Resultant reaction mixture was stirred at 0° C. to approximately 20 to 25° C. in gradient for 2 h. After the reaction was completed, the reaction mixture was quenched by saturated aqueous NH₄Cl solution (250 mL) and was extracted with ethyl acetate (2×500 mL). The organic layer was separated, dried over Na₂SO₄, and was concentrated under reduced pressure to get a crude mass. It was crystallized with isopropanol (100 mL) to get the title compound as a brown solid (4.2 g).

¹H-NMR (300 MHz, DMSO-d₆) δ 8.66 (d, J=2.0 Hz, 1H), 8.35 (d, J=2.0 Hz, 2H), 3.24 (q, J=7.3 Hz, 2H), 1.28 (t, J=7.3 Hz, 3H).

### Step 2: Synthesis of 1-(5-bromo-3-ethylsulfanyl-2-pyridyl) ethanone

To a stirred solution of 5-bromo-3-ethylsulfanyl-pyridine-2-carbonitrile (4.0 g) in dry THF (50 mL) was cooled to 0° C. and followed by dropwise addition of CH₃MgBr (3M in DEE, 10.97 mL). Resultant reaction mixture was stirred at 0° C. for 2 h. After the reaction was completed, the reaction mixture was quenched by saturated aqueous NH₄Cl solution (250 mL) and was extracted with ethyl acetate (2×500 mL). Organic layer was separated, dried over Na₂SO₄, and was concentrated under reduced pressure to get a crude mass. It was purified by column chromatography using ethyl acetate and heptane as eluent to afford the title compound as an off white solid (3.8 g).

¹H-NMR (300 MHz, CDCl₃) δ 8.33 (d, J=1.9 Hz, 1H), 7.69 (d, J=1.9 Hz, 1H), 2.83 (q, J=7.4 Hz, 2H), 2.60 (s, 3H), 1.33 (t, J=7.4 Hz, 3H).

### Step 3: Synthesis of ethyl 3-(5-bromo-3-ethylsulfanyl-2-pyridyl)-3-oxo-propanoate

A stirred solution of 1-(5-bromo-3-ethylsulfanyl-2-pyridyl) ethanone (3.8 g) in heptane (40 mL) was cooled to 0° C., then added NaH 60% (1.75 g) portion wise and followed by the addition of diethyl carbonate (7.2 mL). The reaction mixture was heated at 85° C. for 6 h. After the reaction was completed, the reaction mixture was quenched by ice cold water (150 mL) and was extracted with ethyl acetate (2×250 mL). The organic layer was separated, dried over Na₂SO₃, and was concentrated under reduced pressure to get a crude mass. It was purified by column chromatography using ethyl acetate and heptane as eluent to afford the title compound as yellow liquid (3.0 g).

LC/MS (method A): Rₜ: 2.44 min; m/z=334 (M+2)⁺

¹H-NMR (500 MHz, CDCl₃) δ 8.41 (t, J=1.5 Hz, 1H), 7.80 (d, J=1.9 Hz, 1H), 4.20 (q, J=7.1 Hz, 2H), 4.13 (s, 2H), 2.94 (q, J=7.4 Hz, 2H), 1.44 (t, J=7.4 Hz, 3H), 1.26 (td, J=7.1, 1.0 Hz, 3H).

### Step 4: Synthesis of 5-(5-bromo-3-ethylsulfanyl-2-pyridyl)-1-methyl-2-(trifluoromethyl)pyrazolo[1,5-a]pyrimidin-7-one (I-12)

To a stirred solution of ethyl 3-(5-bromo-3-ethylsulfanyl-2-pyridyl)-3-oxo-propanoate (3 g) and 1-methyl-5-(trifluoromethyl)pyrazol-3-amine (2.0 g) in 1,4-dioxane (1.0 mL) was added polyphosphoric acid (2.0 mL) at 20 to 25° C. The reaction mixture was heated to 130° C. for 5 h. The progress of the reaction was monitored by LCMS analysis. After the reaction was completed, the reaction mixture was quenched by 1N NaOH solution (150 mL) and extracted ethyl acetate (2×100 mL). Combined organic layers were dried over Na₂SO₄ and concentrated under reduced pressure. The crude product was purified by column chromatography using ethyl acetate and heptane as eluent to afford the desired product as an off white solid (1.5 g).

¹H-NMR (500 MHz, DMSO-d₆) δ 8.53 (d, J=2.2 Hz, 1H), 8.07 (d, J=2.2 Hz, 1H), 7.43 (s, 1H), 6.48 (d, J=1.9 Hz, 1H), 4.35 (s, 3H), 3.01 (q, J=7.5 Hz, 2H), 1.20 (dd, J=8.3, 6.4 Hz, 3H).

### Step 5: Synthesis of 5-(5-bromo-3-ethylsulfonyl-2-pyridyl)-1-methyl-2-(trifluoromethyl)pyrazolo[1,5-a]pyrimidin-7-one (I-1)

A stirred solution of 5-(5-bromo-3-ethylsulfanyl-2-pyridyl)-1-methyl-2-(trifluoromethyl)pyrazolo[1,5-a]pyrimidin-7-one (1.5 g) in DCM (20 mL) was cooled to 0° C. and was added meta-chloroperoxybenzoic acid (1.54 g) portion wise. The reaction mixture was stirred at 20 to 25° C. for 16 h. After the reaction was completed, the reaction mixture was quenched by sat. Na₂S₂O₃ solution (70 mL) and extracted with ethyl acetate (2×100 mL). Combined organic layers were dried over Na₂SO₄ and concentrated under reduced pressure. The crude product was purified by column chromatography using ethyl acetate and heptane as eluent to afford the title compound as an off white solid (0.9 g).

¹H-NMR (500 MHz, DMSO-d₆) δ 9.6 (d, J=2.3 Hz, 1H), 8.6 (dd, J=8.2, 2.4 Hz, 1H), 7.57 (dd, J=8.2, 2.1 Hz, 1H), 6.4 (d, J=2.0 Hz, 1H), 4.37 (d, J=1.9 Hz, 3H), 3.80-3.71 (m, 2H), 1.19 (td, J=7.4, 2.1 Hz, 3H).

### Example 2: Synthesis of 5-[5-[[dimethyl(oxo)-λ6-sulfanylidene]amino]-3-ethylsulfonyl-2-pyridyl]-1-methyl-2-(trifluoromethyl)pyrazolo[1,5-a]pyrimidin-7-one (Compound I-2)

To a stirred solution of 5-(5-bromo-3-ethylsulfonyl-2-pyridyl)-1-methyl-2-(trifluoromethyl)pyrazolo[1,5-a]pyrimidin-7-one (0.15 g) in 1,4-dioxane (2 mL) were added K₂CO₃ (0.157 g), dimethyl sulfoximine (0.032 g) and xanthphos (0.018 g), the reaction mixture was degassed under nitrogen for 10 min and then followed by the addition of Pd₂(dba)₃ (0.015 g). The reaction mixture was heated to 120° C. for 5 h. After the reaction was completed, the reaction mixture was concentrated under reduced pressure. The crude product was purified by column chromatography using methanol and DCM as eluent to afford the title compound as an off white solid (0.1 g).

¹H-NMR (300 MHz, DMSO-d₆) δ 8.45 (d, J=2.5 Hz, 1H), 7.87 (d, J=2.5 Hz, 1H), 7.46 (s, 1H), 6.36 (s, 1H), 4.36 (s, 3H), 3.93 (t, J=7.4 Hz, 2H), 2.96 (s, 6H), 1.24 (t, J=7.4 Hz, 3H).

### Example 3: Synthesis of 5-[3-ethylsulfonyl-5-(4-fluorophenyl)-2-pyridyl]-1-methyl-2-(trifluoro-methyl)pyrazolo[1,5-a]pyrimidin-7-one (I-3)

To a stirred solution of 5-(5-bromo-3-ethylsulfonyl-2-pyridyl)-1-methyl-2-(trifluoromethyl)pyrazolo[1,5-a]pyrimidin-7-one (0.15 g) in 1,4-dioxane (2 mL) were added K₂CO₃ (0.134 g) and 4-fluorophenyl boronic acid (0.09 g), the reaction mixture was degassed under nitrogen for 10 min and then followed by the addition of 1,1′-bis(diphenylphosphino)ferrocene]dichloropalladium(II) (0.019 g). The reaction mixture was heated to 120° C. for 5 h. After the reaction was completed, the reaction mixture was concentrated on reduced pressure. The crude product was purified by column chromatography using ethyl acetate and heptane as eluent to afford the title compound as an off white solid (0.1 g).

¹H-NMR (300 MHz, DMSO-d₆) δ 9.27 (d, J=2.2 Hz, 1H), 8.56 (d, J=2.2 Hz, 1H), 7.96 (dd, J=8.6, 5.3 Hz, 2H), 7.51 (s, 1H), 7.43 (t, J=8.8 Hz, 2H), 6.45 (s, 1H), 4.40 (s, 3H), 3.95 (q, J=7.4 Hz, 2H), 1.29 (t, J=7.4 Hz, 3H).

### Example 4: Synthesis of 5-[5-[4-(difluoromethoxy)phenyl]-3-ethylsulfonyl-2-pyridyl]-1-methyl-2-(trifluoromethyl)pyrazolo[1,5-a]pyrimidin-7-one (I-4)

To a stirred solution of 5-(5-bromo-3-ethylsulfonyl-2-pyridyl)-1-methyl-2-(trifluoromethyl)pyrazolo[1,5-a]pyrimidin-7-one (0.15 g) in 1,4-dioxane (2 mL) were added K₂CO₃ (0.134 g) and 2-[4-(difluoromethoxy)phenyl]-4,4,5,5-tetramethyl-1,3,2-dioxaborolane (0.09 g), the reaction mixture was degassed under nitrogen for 10 min and then followed by the addition of bis(diphenylphosphino)ferrocene]dichloropalladium (II) (0.019 g). The reaction mixture was heated to 120° C. for 5 h. After the reaction was completed, the reaction mixture was concentrated under reduced pressure. The crude product was purified by column chromatography using ethyl acetate and heptane as eluent to afford the title compound as an off white solid (0.1 g).

¹H-NMR (300 MHz, DMSO-d₆) δ 9.36 (d, J=2.2 Hz, 1H), 8.65 (d, J=2.2 Hz, 1H), 8.18-8.02 (m, 2H), 7.62 (m, 1H), 7.52 (s, 1H), 7.3 (m, 2H), 6.46 (s, 1H), 4.40 (s, 3H), 3.96 (q, J=7.4 Hz, 2H), 1.28 (q, J=8.7, 8.1 Hz, 3H).

### Example 5: Synthesis of 5-(4-bromo-2-ethylsulfonyl-phenyl)-1-methyl-2-(trifluoromethyl)pyrazolo[1,5-a]pyrimidin-7-one (I-7)

### Step 1: Synthesis of 1-(4-bromo-2-ethylsulfanyl-phenyl)ethanone

A stirred solution of 1-(4-bromo-2-fluoro-phenyl)ethanone (4.0 g) in dry THF (50 mL) was cooled to 0° C., were added NaSC₂H₅ (2.2 g) and 18-crown-6-ether catalytic amount (0.05 g). The reaction mixture was stirred at 20 to 25° C. for 16 h. After the reaction was completed, the reaction mixture was quenched by saturated aqueous NH₄Cl solution (250 mL) and was extracted with ethyl acetate (2×500 mL). Organic layer was separated, dried over Na₂SO₄, and was concentrated under reduced pressure. The crude product was purified by column chromatography using ethyl acetate and heptane as eluent to afford the title compound as an off white solid (3.8 g).

¹H-NMR (300 MHz, DMSO-d₆) δ 7.89 (d, J=8.3 Hz, 1H), 7.53 (d, J=1.9 Hz, 1H), 7.46 (dd, J=8.3, 1.9 Hz, 1H), 2.95 (q, J=7.4 Hz, 3H), 2.55 (s, 3H), 1.25 (t, J=7.3 Hz, 3H).

Step 2: Synthesis of ethyl 3-(4-bromo-2-ethylsulfanyl-phenyl)-3-oxo-propanoate A stirred solution of 1-(4-bromo-2-ethylsulfanyl-phenyl)ethanone (3.8 g) in heptane (40 mL) was cooled to 0° C. and was added NaH 60% (1.75 g) portion wise and then followed by the addition of diethyl carbonate (7.2 mL). The reaction mixture was heated at 85° C. for 6 h. After the reaction was completed, the reaction mixture was quenched by ice cold water (150 mL) and was extracted with ethyl acetate (2×250 mL). The organic layer was separated, dried over Na₂SO₄, and was concentrated under reduced pressure. The crude product was purified by column chromatography using ethyl acetate and heptane as eluent to afford the title compound as yellow liquid (3 g).

LC/MS (method A): Rₜ: 2.42 min; m/z=334 (M+2)⁺

¹H-NMR (300 MHz, CDCl₃) δ 7.64 (d, J=8.4 Hz, 1H), 7.51 (d, J=1.8 Hz, 1H), 7.40-7.34 (m, 1H), 4.22 (q, J=7.0 Hz, 2H), 3.99 (s, 2H), 2.96 (q, J=7.5 Hz, 2H), 1.35-1.21 (m, 3H), 0.88 (dd, J=8.1, 5.3 Hz, 3H).

### Step 3: Synthesis of 5-(4-bromo-2-ethylsulfanyl-phenyl)-1-methyl-2-(trifluoromethyl)pyrazolo[1,5-a]pyrimidin-7-one was Achieved by Previous Method

LC/MS (method A): Rₜ: 2.22 min; m/z=433 (M+1)⁺

¹H-NMR (500 MHz, DMSO-d₆) δ 7.57 (d, J=2.5 Hz, 1H), 7.45 (d, J=8.1 Hz, 1H), 7.40 (s, 1H), 7.37 (dd, J=8.3, 1.9 Hz, 1H), 6.25 (d, J=2.0 Hz, 1H), 4.32 (s, 3H), 2.99 (dd, J=7.3, 1.9 Hz, 2H), 1.19 (td, J=7.4, 2.1 Hz, 3H).

### Step 4: Synthesis of 5-(4-bromo-2-ethylsulfonyl-phenyl)-1-methyl-2-(trifluoromethyl)pyrazolo[1,5-a]pyrimidin-7-one (I-7)

A stirred solution of 5-(4-bromo-2-ethylsulfanyl-phenyl)-1-methyl-2-(trifluoromethyl)pyrazolo[1,5-a]pyrimidin-7-one (1.5 g) in DCM (20 mL) was cooled to 0° C. and was added meta-chloroperoxybenzoic acid (1.54 g) portion wise. The reaction mixture was stirred at 20 to 25° C. for 16 h. After the reaction was completed, the reaction mixture was quenched by sat. Na₂S₂O₃ solution (70 mL) and extracted with ethyl acetate (2×100 mL). The combined organic layers were dried over Na₂SO₄ and concentrated under reduced pressure. The crude product was purified by column chromatography using ethyl acetate and heptane as eluent to afford the title compound as an off white solid (0.9 g).

¹H-NMR (500 MHz, DMSO-d₆) δ 8.11 (d, J=2.3 Hz, 1H), 8.06 (dd, J=8.2, 2.4 Hz, 1H), 7.57 (dd, J=8.2, 2.1 Hz, 1H), 7.45 (d, J=2.0 Hz, 1H), 6.29 (d, J=2.0 Hz, 1H), 4.37 (d, J=1.9 Hz, 3H), 3.80-3.71 (m, 2H), 1.19 (td, J=7.4, 2.1 Hz, 3H).

### Example 5: Synthesis of 5-(5-bromo-3-ethylsulfonyl-2-pyridyl)-2-cyclopropyl-1-methyl-pyrazolo[1,5-a]pyrimidin-7-one (I-10)

### Step 1: Synthesis of 5-cyclopropyl-1-methyl-pyrazol-3-amine

To a stirred solution of 5-cyclopropyl-1H-pyrazol-3-amine (2 g) in DCM (20 mL) were added tBuOK (2.7 g) and CH₃I (0.103 mL) dropwise at 0° C. The reaction mixture was stirred at 20 to 25° C. for 3 h. After the reaction was completed, reaction mixture was diluted with water (150 mL) then extracted with ethyl acetate (2×150 mL). Combined organic layers were dried over Na₂SO₄ and concentrated under reduced pressure. The crude product was purified by column chromatography using ethyl acetate and heptane as eluent to obtain the title compound as brown liquid (1.5 g).

LC/MS (method A): Rₜ: 0.789 min; m/z=138 (M+1)⁺

¹H-NMR (500 MHz, CDCl₃) δ 5.10 (dd, J=7.0, 1.7 Hz, 1H), 3.61 (d, J=1.7 Hz, 3H), 3.49 (d, J=1.8 Hz, 2H), 1.48 (s, 1H), 0.91-0.80 (m, 2H), 0.64-0.47 (m, 2H).

### Step 2: Synthesis of 5-(5-bromo-3-ethylsulfanyl-2-pyridyl)-2-cyclopropyl-1-methyl-pyrazolo[1,5-a]pyrimidin-7-one

To a stirred solution of ethyl 3-(5-bromo-3-ethylsulfanyl-2-pyridyl)-3-oxo-propanoate (0.7 g) and 5-cyclopropyl-1-methyl-pyrazol-3-amine (0.434 g) in 1,4-dioxane (0.5 mL) was added PPA (2 mL) at 20 to 25° C. The reaction mixture was heated to 130° C. for 5 h. After the reaction was completed, reaction mixture was quenched by 1N NaOH solution (50 mL) and extracted with ethyl acetate (2×75 mL). Combined organic layers were dried over Na₂SO₄ and concentrated under reduced pressure. The crude product was purified by column chromatography using ethyl acetate and heptane as eluent to afford the title compound as an off white solid (0.5 g).

LC/MS (method A): Rₜ: 2.16 min; m/z=406 (M+1)⁺

¹H-NMR (500 MHz, DMSO-d₆) δ 8.27 (d, J=2.4 Hz, 1H), 7.81 (d, J=2.8 Hz, 1H), 6.06 (d, J=2.1 Hz, 1H), 5.97 (d, J=2.0 Hz, 1H), 4.10 (d, J=2.0 Hz, 3H), 2.76 (dd, J=7.5, 1.9 Hz, 2H), 1.78 (d, J=2.1 Hz, 1H), 1.18 (t, J=7.4 Hz, 3H), 1.05-0.82 (m, 2H), 0.78-0.69 (m, 2H).

### Step 3: Synthesis of 5-(5-bromo-3-ethylsulfonyl-2-pyridyl)-2-cyclopropyl-1-methyl-pyrazolo[1,5-a]pyrimidin-7-one

A stirred solution of 5-(5-bromo-3-ethylsulfanyl-2-pyridyl)-2-cyclopropyl-1-methyl-pyrazolo[1,5-a]pyrimidin-7-one (0.5 g) in DCM (10 mL) was cooled to 0° C. and was added meta-chloroperoxybenzoic acid (0.54 g). The reaction mixture was stirred at 20 to 25° C. for 16 h. After the reaction was completed, reaction mixture was quenched by sat. Na₂S₂O₃ solution (70 mL) and extracted with ethyl acetate (2×100 mL). Combined organic layers were dried over Na₂SO₄ and concentrated under reduced pressure. The crude product was purified by column chromatography using ethyl acetate and heptane as eluent to afford the title compound as an off white solid (0.3 g).

¹H-NMR (500 MHz, DMSO-d₆) δ 9.09 (s, 1H), 8.51 (s, 1H), 6.31 (s, 1H), 6.09 (d, J=1.8 Hz, 1H), 4.35 (s, 3H), 3.94 (q, J=7.5 Hz, 2H), 2.12 (s, 1H), 1.23 (t, J=7.4 Hz, 3H), 1.15 (d, J=8.0 Hz, 2H), 0.96 (d, J=5.3 Hz, 2H).

### Example 6: Synthesis of 5-[5-bromo-3-(ethylsulfonimidoyl)-2-pyridyl]-1-methyl-2-(trifluoromethyl)pyrazolo[1,5-a]pyrimidin-7-one (I-13)

A stirred solution of 5-(5-bromo-3-ethylsulfanyl-2-pyridyl)-1-methyl-2-(trifluoromethyl)pyrazolo[1,5-a]pyrimidin-7-one (0.12 g) in methanol (50 mL) was cooled to 0° C. and were added ammonium carbamate (0.022 g) and (diacetoxyiodo)benzene (0.089 g). The reaction mixture was stirred at 20 to 25° C. for 2 h. After the reaction was completed, the reaction mixture was concentrated under reduced pressure. The crude product was purified by column chromatography using methanol and DCM as eluent to afford the title compound as an off white solid (0.09 g).

¹H-NMR (500 MHz, DMSO-d₆) δ 9.02 (s, 1H), 8.54 (s, 1H), 7.46 (s, 1H), 6.30 (s, 1H), 4.43 (s, 1H), 4.37 (s, 3H), 3.70 (q, J=7.4 Hz, 2H), 1.19 (t, J=7.4 Hz, 3H).

### Example 7: Synthesis of 5-(5-bromo-3-ethylsulfonyl-2-pyridyl)-1,6-dimethyl-2-(trifluoromethyl)pyrazolo[1,5-a]pyrimidin-7-one (I-16)

### Step 1: Synthesis of ethyl 3-(5-bromo-3-ethylsulfanyl-2-pyridyl)-2-methyl-3-oxo-propanoate

To a stirred solution of ethyl 3-(5-bromo-3-ethylsulfanyl-2-pyridyl)-3-oxo-propanoate (1.2 g) in MeCN (10 mL) were added K₂CO₃ (0.75 g) and CH₃I (0.22 mL) at 0° C. The reaction mixture was heated to 50° C. for 3 h. After the reaction was completed, the reaction mixture was diluted with water (150 mL) then extracted with ethyl acetate (2×150 mL). Combined organic layers were dried over Na₂SO₄ and concentrated under reduced pressure. The crude product was purified by column chromatography using ethyl acetate and heptane as eluent to obtain the title compound as brown liquid (0.9 g).

LC/MS (method A): Rₜ: 2.54 min; m/z=344 (M-2)⁺

¹H-NMR (500 MHz, CDCl₃) δ 8.41 (t, J=1.5 Hz, 1H), 7.80 (d, J=1.9 Hz, 1H), 4.6 (t, J=8.2 Hz, 1H), 4.20 (d, J=7.1 Hz, 2H), 2.94 (q, J=7.4 Hz, 2H), 1.44 (t, J=7.4 Hz, 6H), 1.26 (td, J=7.1, 1.0 Hz, 3H).

### Step 2: Synthesis of 5-(5-bromo-3-ethylsulfanyl-2-pyridyl)-1,6-dimethyl-2-(trifluoromethyl)pyrazolo[1,5-a]pyrimidin-7-one was Achieved Following Previous Method

LC/MS (method A): Rₜ: 2.32 min; m/z=448 (M+1)⁺

¹H-NMR (300 MHz, DMSO-d₆) δ 8.60-8.48 (m, 1H), 8.13 (d, J=2.0 Hz, 1H), 7.35 (s, 1H), 4.27 (s, 3H), 3.04 (q, J=7.3 Hz, 2H), 1.18 (t, J=7.2 Hz, 3H), 1.26 (td, J=7.1, 1.0 Hz, 3H).

### Step 3: Synthesis of 5-(5-bromo-3-ethylsulfonyl-2-pyridyl)-1,6-dimethyl-2-(trifluoromethyl)pyrazolo[1,5-a]pyrimidin-7-one (I-16)

A stirred solution of 5-(5-bromo-3-ethylsulfanyl-2-pyridyl)-1,6-dimethyl-2-(trifluoromethyl)pyrazolo[1,5-a]pyrimidin-7-one (0.5 g) in DCM (10 mL) was cooled to 0° C. and was added meta-chloroperoxybenzoic acid (0.54 g). The reaction mixture was stirred at 20 to 25° C. for 16 h. The progress of the reaction was monitored by TLC. After the reaction was completed, reaction mixture was quenched by sat. Na₂S₂O₃ solution (70 mL) and extracted with ethyl acetate (2×100 mL). Combined organic layers were dried over Na₂SO₄ and concentrated under reduced pressure. The crude product was purified by column chromatography using ethyl acetate and heptane as eluent to afford the title compound as an off white solid (0.1 g).

¹H-NMR (500 MHz, DMSO-d₆) δ 9.15 (s, 1H), 8.58 (s, 1H), 7.39 (s, 1H), 4.30 (s, 3H), 3.61 (d, J=7.4 Hz, 2H), 1.82 (s, 3H), 1.17 (t, J=7.3 Hz, 3H).

With appropriate modification of the starting materials, the procedures given in the synthesis descriptions were used to obtain further compounds I. The compounds obtained in this manner are listed in the table below, together with physical data.

TABLE I
compounds of formula I.X

| | |
|---|---|
| | I.X |
| 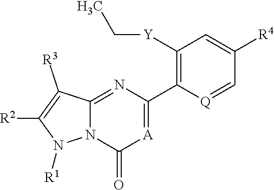 | |

| | | | | | | | | phys. data (HPLC) | | |
|---|---|---|---|---|---|---|---|---|---|---|
| | | | | | | | | | | M + H |
| No. | R¹ | R² | R³ | A | Y | Q | R⁴ | Method | Rₜ [min] | [m/z] |
| I-1 | CH₃ | CF₃ | H | CH | SO₂ | N | Br | A | 1.72 | 466 |
| I-2 | CH₃ | CF₃ | H | CH | SO₂ | N | N═S(O)(CH₃)₂ | A | 1.503 | 478 |
| I-3 | CH₃ | CF₃ | H | CH | SO₂ | N | 4-F—C₆H₄ | A | 2.01 | 481 |
| I-4 | CH₃ | CF₃ | H | CH | SO₂ | N | 4-OCHF₂—C₆H₄ | A | 2.24 | 530 |
| I-5 | CH₃ | CF₃ | H | CH | SO₂ | N | 4-CN—C₆H₄ | A | 2.059 | 488 |
| I-6 | CH₃ | CF₃ | H | CH | SO₂ | N | 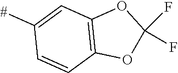 | A | 2.12 | 543 |
| I-7 | CH₃ | CF₃ | H | CH | SO₂ | CH | Br | A | 2.147 | 465 |
| I-8 | CH₃ | CF₃ | H | CH | SO₂ | CH | 4-F—C₆H₄ | A | 2.34 | 481 |
| I-9 | CH₃ | CF₃ | H | CH | SO₂ | CH | N═S(O)(CH₃)₂ | A | 1.572 | 477 |
| I-10 | CH₃ | cC₃H₅ | H | CH | SO₂ | N | Br | A | 1.88 | 438 |
| I-11 | CH₃ | cC₃H₅ | H | CH | SO₂ | N | 4-F—C₆H₄ | A | 2.113 | 453 |
| I-12 | CH₃ | CF₃ | H | CH | S | N | Br | A | 2.322 | 434 |
| I-13 | CH₃ | CF₃ | H | CH | S(O)═NH | N | Br | A | 1.76 | 465 |
| I-14 | CH₃ | CF₃ | H | CH | SO₂ | N | 2-CN-pyrid-5-yl | A | 1.88 | 489 |
| I-15 | CH₃ | CF₃ | H | CH | SO₂ | N | 2-F-pyrid-5-yl | A | 1.934 | 482 |
| I-16 | CH₃ | CF₃ | H | CCH₃ | SO₂ | N | Br | A | 2.082 | 480 |
| I-17 | CH₃ | H | H | CH | SO₂ | N | Br | A | 1.558 | 398 |
| I-18 | CH₃ | cC₃H₅ | H | CH | SO₂ | N | 4-OCF₃-2-F—C₆H₃ | A | 2.16 | 537 |
| I-19 | CH₃ | cC₃H₅ | H | CH | SO₂ | N | 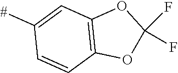 | A | 2.12 | 515 |
| I-20 | CH₃ | cC₃H₅ | H | CBr | SO₂ | N | 4-OCF₃-2-F—C₆H₃ | A | 2.20 | 616 |
| I-21 | CH₃ | H | 4-F—C₆H₄ | CH | SO₂ | N | H | A | 1.81 | 413 |

# denotes the bond to the remainder of the molecule

| Compound | |
|---|---|
| No. | δ [ppm] |
| I-5 | (300 MHz, DMSO-d₆) 9.36 (d, J = 2.2 Hz, 1H), 8.65 (d, J = 2.2 Hz, 1H), 8.18-8.02 |
| | (m, 4H), 7.52 (s, 1H), 6.46 (s, 1H), 4.4 (s, 3H), 3.96 (q, J = 7.4 Hz, 2H), 1.30 (t, |
| | J = 7.4 Hz, 3H) |
| I-6 | (300 MHz, DMSO-d₆) 9.27 (d, J = 2.2 Hz, 1H), 8.58 (d, J = 2.2 Hz, 1H), 8.05 (d, |
| | J = 1.8 Hz, 1H), 7.81-7.71 (m, 1H), 7.63 (d, J = 8.5 Hz, 1H), 7.50 (s, 1H), 6.45 (s, |
| | 1H), 4.40 (s, 3H), 3.94 (q, J = 7.4 Hz, 2H), 1.28 (t, J = 7.4 Hz, 3H) |
| I-8 | (300 MHz, DMSO-d₆) δ 8.20 (d, J = 1.9 Hz, 1H), 8.11 (dd, J = 7.9, 1.9 Hz, 1H), 7.85 |
| | (dd, J = 8.7, 5.3 Hz, 2H), 7.70 (d, J = 7.9 Hz, 1H), 7.41 (dd, J = 17.5, 8.8 Hz, 3H), |
| | 6.32 (s, 1H), 4.38 (s, 3H), 3.78 (q, J = 7.4 Hz, 2H), 1.25 (t, J = 7.3 Hz, 3H) |
| I-9 | (500 MHz, DMSO-d₆) δ 7.52 (s, 1H), 7.41 (d, J = 7.1 Hz, 2H), 7.29 (d, J = 8.2 Hz, |
| | 1H), 6.20 (s, 1H), 4.34 (s, 3H), 3.72 (q, J = 7.4 Hz, 2H), 2.96 (s, 6H), 1.18 (t, |
| | J = 7.4 Hz, 3H) |
| I-11 | (300 MHz, DMSO-d₆) δ 9.2 (s, 1H), 8.5 (s, 1H), 7.93 (dd, J = 8.7, 5.3 Hz, 2H), |
| | 7.42 (d, J = 7.9 Hz, 2H), 6.3 (s, 1 H), 6.1 (s, 1H), 4.35 (s, 3H), 3.95 (q, J = 7.4 Hz, |
| | 2H), 2.12 (s, 1H), 1.25 (t, J = 7.3 Hz, 3H), 1.15 (d, J = 8.0 Hz, 2H), 0.96 (d, |
| | J = 5.3 Hz, 2H) |
| I-14 | (500 MHz, DMSO-d₆) δ 9.41 (d, J = 2.3 Hz, 1H), 9.31 (s, 1H), 8.75 (d, J = 2.3 Hz, |
| | 1H), 8.27 (d, J = 8.1 Hz, 1H), 7.52 (s, 1H), 6.46 (d, J = 1.8 Hz, 1H), 5.76 (t, |
| | J = 1.3 Hz, 1H), 4.40 (s, 3H), 3.95 (q, J = 3 Hz, 2H), 1.30 (dt, J = 8.5, 4.2 Hz, 3H). |
| I-15 | (500 MHz, DMSO-d₆) δ 9.33 (d, J = 2.4 Hz, 1H), 8.79 (s, 1H), 8.65 (s, 1H), 8.54 (t, |
| | J = 8.2 Hz, 1H), 7.51 (s, 1H), 7.43 (d, J = 8.5 Hz, 1H), 6.45 (s, 1H), 4.40 (s, 3H), |
| | 3.94 (q, J = 7.4 Hz, 2H), 1.29 (t, J = 7.4 Hz, 3H) |
| I-17 | (500 MHz, DMSO-d₆) δ 9.10 (d, J = 2.1 Hz, 1H), 8.53 (d, J = 2.1 Hz, 1H), 8.35 (t, |
| | J = 2.6 Hz, 1H), 6.57 (dd, J = 3.4, 1.9 Hz, 1H), 6.12 (d, J = 1.9 Hz, 1H), 4.36 (d, |
| | J = 1.9 Hz, 3H), 3.95 (qd, J = 7.4, 1.8 Hz, 2H), 1.26-1.16 (m, 3H) |
| I-18 | (500 MHz, DMSO-d₆) δ 8.13 (t, J = 1.6 Hz, 1H), 8.00 (dd, J = 7.8, 1.8 Hz, 1H), 7.82 |
| | (q, J = 8.2, 7.7 Hz, 1H), 7.62 (d, J = 11.0 Hz, 1H), 7.44 (d, J = 8.6 Hz, 1H), 6.27 (d, |
| | J = 0.6 Hz, 1H), 6.00 (s, 1H), 4.33 (s, 3H), 3.78 (q, J = 7.3 Hz, 2H), 2.14 (dd, J = 8.8, |
| | 4.0 Hz, 1H), 1.21-1.23 (t, J = 7.4 Hz, 3H), 1.08 (dt, J = 6.8, 4.7 Hz, 2H). 0.96 (dt, |
| | J = 6.8, 4.7 Hz, 2H) |
| I-20 | (300 MHz, DMSO-d₆) δ 8.13 (s, 1H), 8.04 (d, J = 8.1 Hz, 1H), 7.86 (t, J = 8.7 Hz, |
| | 1H), 7.62 (d, J = 8.2 Hz, 1H), 7.44 (d, J = 8.7 Hz, 1H), 6.31 (s, 1H), 4.34 (s, 3H), |
| | 3.65-3.39 (m, 2H), 2.14 (dd, J = 8.8, 4.0 Hz, 1H), 1.19-1.08 (m, 3H), 0.97 (d, |
| | J = 13.3 Hz, 2H), 0.93 (s, 2H) |
| I-21 | (500 MHz, DMSO-d₆) δ 9.24 (s, 1H), 8.54 (d, J = 2.7 Hz, 1H), 8.36 (d, J = 2.9 Hz, |
| | 1H), 7.95 (t, J = 6.7 Hz, 2H), 7.43 (t, J = 8.4 Hz, 2H), 6.57 (d, J = 3.0 Hz, 1H), 6.16 |
| | (s, 1H), 4.37 (s, 3H), 3.97 (q, J = 7.3 Hz, 2H), 1.28 (t, J = 7.6 Hz, 3H) |

### BIOLOGICAL EXAMPLES

If not otherwise specified, the test solutions were prepared as follow:

The active compound was dissolved at the desired concentration in a mixture of 1:1 (vol:vol) distilled water:acetone. The test solution was prepared on the day of use.

The activity of the compounds of formula I of the present invention can be demonstrated and evaluated by the following biological tests.

B.1 Green Peach Aphid (Myzus persicae)

For evaluating control of green peach aphid (Myzus persicae) through systemic means, the test unit consisted of 96-well-microtiter plates containing liquid artificial diet under an artificial membrane.

The compounds were formulated using a solution containing 75% v/v water and 25% v/v DMSO. Different concentrations of formulated compounds were pipetted into the aphid diet, using a custom built pipetter, at two replications.

After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed.

In this test, compounds I-1, I-2, I-3, I-4, I-5, I-6, I-7, I-8, I-9, I-11, I-14, I-15, I-16 and I-21 resp., at 2500 ppm showed at least 75% mortality in comparison with untreated controls.

B.2 Tobacco Budworm (Heliothis virescens)

For evaluating control of tobacco budworm (Heliothis virescens), the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom-built micro atomizer, at two replications.

After application, microtiter plates were incubated at about 28±1° C. and about 80±5% relative humidity for 5 days. Egg and larval mortality was then visually assessed.

In this test, compounds I-1, I-3, I-4, I-5, I-6, I-8, I-9, I-11, I-14 and I-15 resp., at 2500 ppm showed at least 75% mortality in comparison with untreated controls.

B.3 Boll Weevil (Anthonomus grandis)

For evaluating control of boll weevil (Anthonomus grandis), the test unit consisted of 96-well-microtiter plates containing an insect diet and 5-10 A. grandis eggs.

The compounds were formulated using a solution containing 75% v/v water and 25% v/v DMSO. Different concentrations of formulated compounds were sprayed onto the insect diet at 5 μl, using a custom-built micro atomizer, at two replications.

After application, microtiter plates were incubated at about 25±1° C. and about 75±5% relative humidity for 5 days. Egg and larval mortality was then visually assessed.

In this test, compounds I-1, I-2, I-3, I-4, I-5, I-6, I-8, I-11, I-14, I-15, I-16 and I-21 resp., at 2500 ppm showed at least 75% mortality in comparison with untreated controls.

B.4. Southern Armyworm (Spodoptera eridania), 2nd Instar Larvae

The active compounds were formulated by a Tecan liquid handler in 100% cyclohexanone as a 10,000-ppm solution supplied in tubes. The 10,000-ppm solution was serially diluted in 100% cyclohexanone to make interim solutions. These served as stock solutions for which final dilutions were made by the Tecan in 50% acetone:50% water (v/v) into 10 or 20 ml glass vials. A non-ionic surfactant (Kinetic®) was included in the solution at a volume of 0.01% (v/v). The vials were then inserted into an automated electrostatic sprayer equipped with an atomizing nozzle for application to plants/insects. Lima bean plants (variety Sieva) were grown 2 plants to a pot and selected for treatment at the 1st true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. Ten to 11 armyworm larvae were placed into the bag and the bags zipped closed. Test plants were maintained in a growth room at about 25° C. and about 20-40% relative humidity for 4 days, avoiding direct exposure to fluorescent light (14:10 light:dark photoperiod) to prevent trapping of heat inside the bags. Mortality and reduced feeding were assessed 4 days after treatment, compared to untreated control plants.

In this test, compounds I-3, I-4, I-5, I-6, I-7, I-8, I-11, I-14 and I-15 resp., at 300 ppm at least 75% mortality in comparison with untreated controls.

B.5 Yellow Fever Mosquito (Aedes aegypti)

For evaluating control of yellow fever mosquito (Aedes aegypti) the test unit consisted of 96-well-microtiter plates containing 200 μl of tap water per well and 5-15 freshly hatched A. aegypti larvae.

The active compounds were formulated using a solution containing 75% (v/v) water and 25% (v/v) DMSO. Different concentrations of formulated compounds or mixtures were sprayed onto the insect diet at 2.5 μl, using a custom-built micro atomizer, at two replications.

After application, microtiter plates were incubated at 28±1° C., 80±5% RH for 2 days. Larval mortality was then visually assessed.

In this test, compounds I-1, I-2, I-3, I-4, I-5, I-6, I-7, I-8, I-14 and I-15 resp., at 2500 ppm showed at least 75% mortality in comparison with untreated controls.

1. A compound of formula I 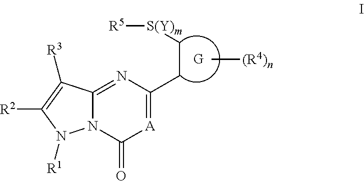
wherein
R¹ is C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkyl-C₁-C₄-alkyl, or C₃-C₆-cycloalkoxy-C₁-C₄-alkyl, which are unsubstituted or halogenated;
phenyl or benzyl, wherein the rings are unsubstituted or substituted with R^{F};
R^{F} is halogen, OH, CN, NO₂, SCN, SF₅, C₁-C₆ alkyl, C₁-C₆ alkoxy, C₂-C₆-alkenyl, C₂-C₆ alkynyl, C₁-C₆ alkoxy-C₁-C₄ alkyl, C₁-C₆ alkoxy-C₁-C₄ alkoxy, C₃-C₆ cycloalkyl, C₃-C₆-cycloalkoxy, C₃-C₆ cycloalkyl-C₁-C₄ alkyl, C₃-C₆-cycloalkoxy-C₁-C₄ alkyl, which groups are unsubstituted or substituted with halogen;
R² is H, halogen, C₁-C₆-alkyl, C₂-C₆-alkenyl, C₂-C₆-alkynyl, C₁-C₆-alkoxy, C₁-C₆-alkoxy-C₁-C₆-alkyl, C₃-C₆-cycloalkyl, C₃-C₆-cycloalkoxy, C₁-C₆-sulfenyl, C₁-C₆-sulfinyl, or C₁-C₆-sulfonyl, which groups are unsubstituted or halogenated; phenyl or benzyl, wherein the rings are unsubstituted or substituted with R^{F};
R³ is H, halogen;
C₁-C₆-alkyl, C₁-C₆-alkoxy, C₃-C₆-cycloalkyl, which are unsubstituted or substituted with R^{F}; or
phenyl or benzyl, wherein the aromatic ring of the aforementioned groups may be unsubstituted or substituted with R^{F};
G is phenyl or 6-membered hetaryl;
R⁴ is H, halogen, C(CN)R⁴¹R⁴², C(R⁴⁴)═N—OR⁴³, C(R⁴⁴)═N—N(R⁴⁵R⁴⁶) C(O)R⁴⁴, N═S(O)(R⁴¹R⁴²), N(R⁴³)C(O)R⁴⁴, N(R⁴³)C(═N—OR⁴³)R⁴⁴; OC(CN)R⁴¹R⁴², C₁-C₆-alkyl, C₃-C₆-cycloalkyl, C₁-C₆-alkoxy, C₂-C₆-alkenyl, C₂-C₆-alkynyl, phenyl, or six-membered hetaryl, which groups are unsubstituted or substituted with R^{G};
R^{G}H, halogen, CN, NO₂, C₁-C₄-alkyl, C₁-C₄-haloalkyl, C₁-C₄-alkoxy, C₁-C₄-haloalkoxy, N(R¹²R¹³), S(O)ₘ—C₁-C₄-haloalkyl, C₁-C₄-alkylcarbonyl, or C₁-C₄-haloalkylcarbonyl;
or two groups R^{G} bonded to two adjacent atoms form a 4- to 6-membered carbo- or heterocyclic ring which is unsubstituted or partially or fully substituted with R^{F};
R⁴¹, R⁴² are independently H, halogen, CN, C₁-C₆-alkyl, C₁-C₆-haloalkyl, C₃-C₆-cycloalkyl, C₁-C₄-alkoxy, C₁-C₄-alkoxy-C₁-C₄-alkyl, S(O)ₘ—C₁-C₄-alkyl, C₁-C₄-alkyl-S(O)ₘ—C₁-C₄-alkyl, or C₁-C₄-alkoxycarbonyl;
R⁴¹ and R⁴² may also form together with the carbon atom to which they are bound, a C₃-C₆-cycloalkyl which is unsubstituted or substituted with halogen, C₁-C₄-alkyl, C₁-C₄-haloalkyl, C₁-C₄-alkoxy, or C₁-C₄-haloalkoxy;
R⁴³ is H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen and/or CN;
phenyl or benzyl, which groups are unsubstituted or substituted with R^{F};
R⁴⁴ is H, CN, OH, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen;
phenyl or benzyl, which groups are unsubstituted or substituted with R^{F};
R⁴⁵, R⁴⁶ are independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, C₁-C₆-alkyl-carbonyl, C₁-C₆-alkoxy-carbonyl, which groups are unsubstituted or substituted with halogen;
phenyl or benzyl, which groups are unsubstituted or substituted with R^{F};
NR⁴⁵R⁴⁶ may also form an N-bound, saturated 5- to 8-membered heterocycle, which additionally to the nitrogen may have 1 or 2 further heteroatoms selected from O, S(O)ₘ, and N—R′, wherein R′ is H or C₁-C₆-alkyl, and which heterocycle is unsubstituted or substituted with halogen, C₁-C₄-alkyl, C₁-C₄-haloalkyl, C₁-C₄-alkoxy, or C₁-C₄-haloalkoxy;
A is CH, CR^{A}, or N;
R^{A} is halogen, C₁-C₄-alkyl, C₃-C₆-cycloalkyl, C₁-C₆-haloalkyl, C₁-C₆-halocycloalkyl, OR⁴³, S(O)ₘ—R⁴³; wherein rings are unsubstituted or substituted with R⁴²;
m is 0, 1, or 2;
n is 0, 1, or 2;
Y is O or NR^{Y};
R^{Y} is H or C₁-C₄-alkyl;
R⁵ is C₁-C₄-alkyl, C₃-C₆-cycloalkyl, C₃-C₆-cycloalkyl-C₁-C₄-alkyl;
and N-oxides, tautomers, stereoisomers, and agriculturally or veterinarily acceptable salts thereof.

2. The compound of formula I according to claim 1, wherein R¹ is CH₃ or cC₃H₅.

3. The compound of formula I according to claim 1, wherein R² is H, c-C₃H₅ or CF₃.

4. The compound of formula I according to claim 1, wherein R³ is H or phenyl substituted with halogen.

5. The compound of formula I according to claim 1, wherein G is phenyl or pyridyl.

6. The compound of formula I according to claim 1, wherein A is CH, or CR^{A}, wherein R^{A} is C₁-C₃-alkyl or halogen.

7. The compound of formula I according to claim 1, wherein R⁴ is H, halogen, C₁-C₄-alkyl, C₂-C₄-alkenyl, C₂-C₄-alkynyl unsubstituted or substituted with R^{G}; C(CN)R⁴¹R⁴², N═S(O)(R⁴¹R⁴²), or phenyl unsubstituted or substituted with halogen, CN, C₁-C₄-alkyl, C₁-C₄-haloalkyl, C₁-C₄-alkoxy, or C₁-C₄-haloalkoxy.

8. The compound of formula I according to claim 5, wherein n is 1 and R⁴ is in para position to the attachment to the bicyclic scaffold.

9. An agricultural or veterinary composition comprising at least one compound according to claim 1 and/or at least one agriculturally or veterinarily acceptable salt thereof, and at least one inert liquid and/or solid agriculturally or veterinarily acceptable carrier.

10. An agricultural composition for combating animal pests comprising at least one compound as defined in claim 1 and at least one inert liquid and/or solid acceptable carrier and, optionally, at least one surfactant.

11. A method for combating or controlling invertebrate pests, comprising contacting said invertebrate pest or its food supply, habitat or breeding grounds with a pesticidally effective amount of at least one compound as defined in claim 1.

12. A method for protecting growing plants from attack or infestation by invertebrate pests, comprising contacting a plant, or soil or water in which the plant is growing, with a pesticidally effective amount of at least one compound as defined in claim 1.

13. A seed comprising a compound as defined in claim 1, or enantiomers, diastereomers, or salts thereof, in an amount of from 0.1 g to 10 kg per 100 kg of seed.

14. A method for treating or protecting an animal from infestation or infection by invertebrate pests comprising bringing the animal in contact with a pesticidally effective amount of at least one compound of formula I as defined in claim 1, a stereoisomer thereof, and/or at least one veterinarily acceptable salt thereof.
